(19) United States
(12) Patent Application Publication
(10) Pub. No.: US 2024/0325364 A1
(43) Pub. Date: Oct. 3, 2024

(54) COMPOSITIONS FOR USE IN THE TREATMENT OF METABOLIC SYNDROME

(71) Applicant: Nottingham Trent University, Nottinghamshire (GB)

(72) Inventors: Mark Duncan Turner, Nottinghamshire (GB); Alun Christopher Garner, Nottinghamshire (GB)

(21) Appl. No.: 18/578,659

(22) PCT Filed: Jul. 8, 2022
(86) PCT No.: PCT/GB2022/051769

§ 371 Date: Jan. 11, 2024

(30) Foreign Application Priority Data
Jul. 15, 2021 (GB) 2110230.6

(51) Int. Cl. A61K 31/4439; A61K 31/4174; A61K 31/42; A61K 31/443; A61P 3/04

(52) U.S. Cl. CPC A61K 31/4439; A61K 31/4174; A61K 31/42; A61K 31/443; A61P 3/04

(57) ABSTRACT

The invention provides a compound of Formula (I) or a tautomer, isomer, prodrug, metal complex, or pharmaceutically acceptable salt thereof for use in the treatment of a metabolic disorder or for inducing weight loss: wherein R¹ is: H; a straight or branched alkyl; a straight or branched alkyl substituted with at least one moiety selected from the group comprising: halogen, hydroxy, alkoxy, NH₂, mono- or disubstituted amino, thiol, and phosphine; aryl; aralkyl; hydroxy; alkoxy; aryloxy; arylalkoxy; NH₂; mono- or disubstituted amino; or halogen; X is: CH or N; L is absent or present, and when present is: an optionally substituted straight or branched C₁-C₁₀ alkyl, optionally containing one or more rings, and/or optionally containing one or more double bonds; and Ar is: an optionally substituted 5-membered unsaturated heterocyclic ring.

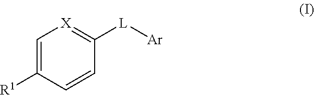

### TECHNICAL FIELD OF THE INVENTION

The present invention relates to compounds for use in the treatment of metabolic disorders or for inducing weight loss, uses of compounds in the manufacture of a medicament for metabolic disorders or for weight loss induction, and to methods of medical treatment comprising the use of compounds to treat metabolic disorders or to induce weight loss.

### BACKGROUND TO THE INVENTION

Being overweight or obese is defined by abnormal or excessive fat accumulation that can present a risk to health. The issue has grown to epidemic proportions, with over 4 million people dying each year as a result of being overweight or obese in 2017 according to the Global Burden of Disease.

Rates of being overweight or obese continue to grow in adults and children. According to the World Health Organisation, from 1975 to 2016, the prevalence of overweight and obese children and adolescents aged 5-19 years increased more than four-fold from 4% to 18% globally. Once considered a problem only in high-income countries, being overweight and/or obese is now also on the rise in low- and middle-income countries, particularly in urban areas.

Being overweight or obese are both major risk factors for a number of chronic diseases, including cardiovascular diseases such as heart disease and stroke, which are the leading causes of death worldwide.

A significant proportion of overweight and obese people also suffer from diabetes, and in particular type-2 diabetes. Obesity is associated with the development of insulin resistance, which results in skeletal muscle cells failing to respond to extracellular insulin and taking insufficient glucose into cells for use as an energy source. Obesity-induced insulin resistance increases demand on the pancreas to release more insulin, which can lead to pancreatic dysfunction as the pancreas struggles to address a sustained demand for increased insulin secretion placed upon it.

Furthermore, the fatty acids associated with obesity combine with glucose and its breakdown products to form damaging non-enzymatic glycation and lipidation end-products that bind to protein, lipid, and DNA, thereby modifying them and preventing normal cellular function.

Previous work indicates that carnosine, a naturally occurring physiological dipeptide, is an effective scavenger of glycation and lipidation end-products, and consequently is able to restore cellular function in key tissues associated with both insulin secretion (pancreatic β-cells) and insulin resistance (skeletal muscle) (Cripps, M. J., Hanna, K., Lavilla, C., Sayers, S. R., Caton, P. W., Sims, C., De Girolamo, L., Sale, C. and Turner, M. D., 2017. Carnosine scavenging of glucolipotoxic free radicals enhances insulin secretion and glucose uptake. Scientific reports, 7(1), pp. 1-7). However, taking carnosine as a supplement is likely to require sustained administration of high doses in order to achieve modest beneficial effects, as there are carnosinase enzymes in both blood and tissues that are able to degrade carnosine.

There exists a need for therapeutics that retain or even expand upon the beneficial biological actions of carnosine, but which also display limited toxicity and resistance to enzymatic degradation, particular by carnosinases.

There also exists a need for compounds and pharmaceutical compositions which can assist or induce weight loss in an obese person, especially those with diabetes or at an increased risk of diabetes.

It is an aim of embodiments of the present invention to address these requirements by providing compounds which provide one or more of the following advantages:
Non-degradability or slow degradability by carnosinase enzymes and/or improved bioavailability.
Ability to be used in the treatment of at least one metabolic disorder.
Ability to be used in the treatment of a weight or dietary-related metabolic disorder.
Ability to be used in the treatment of obesity and/or a glucose-related metabolic disorder, such as insulin resistance which may be obesity-induced.
Ability to assist in regulating blood glucose levels and/or the ability to be used in the treatment of glucose-related metabolic disorders, such as diabetes; and preferably type-2 diabetes.
Ability to be used for assisting/inducing weight loss.
Limited or no toxicity.
Reactive species scavenging ability.
Membrane transport compatibility.

It is also an aim of embodiments of the invention to overcome or mitigate at least one problem of the prior art, whether expressly described herein or not.

### SUMMARY OF THE INVENTION

According to a first aspect of the invention, there is provided a compound of Formula (I) or a tautomer, isomer, prodrug, metal complex, or pharmaceutically acceptable salt thereof for use in the treatment of a metabolic disorder or for inducing weight loss:

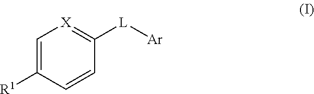

wherein R¹ is:
H; a straight or branched alkyl; a straight or branched alkyl substituted with at least one moiety selected from the group comprising: halogen, hydroxy, alkoxy, NH₂, mono- or disubstituted amino, thiol, and phosphine; aryl; aralkyl; hydroxy; alkoxy; aryloxy; arylalkoxy; NH₂; mono- or disubstituted amino; or halogen;
X is:
CH or N;
L is absent or present, and when present is:
an optionally substituted straight or branched C₁-C₁₀ alkyl, optionally containing one or more rings, and/or optionally containing one or more double bonds; and
Ar is:
an optionally substituted 5-membered unsaturated heterocyclic ring.

Such compounds provide the beneficial biological actions of carnosine, whilst at the same time have modified structures that are not likely to be susceptible to degradation by carnosinase enzymes. Such compounds are suitable for use as therapeutics in the treatment of metabolic disorders, and in particular weight or dietary-related metabolic disorders. Such compounds are especially effective in the treatment of obesity and for inducing weight loss. The compounds are also effective in the treatment of glucose-related metabolic disorders, such as diabetes and insulin resistance which may be obesity-induced.

In some embodiments, R¹ is selected from the group comprising: aryl, aralkyl, hydroxy, alkoxy, aryloxy, arylalkoxy, NH₂, mono- or disubstituted amino, and halogen.

In some embodiments, R¹ is a straight or branched alkyl.

In some embodiments, R¹ is a straight or branched alkyl substituted with at least one moiety selected from the group comprising: halogen, hydroxy, alkoxy, NH₂, mono- or disubstituted amino, thiol, and phosphine.

In some embodiments, R¹ is a straight or branched C₁-C₁₀ alkyl, C₁-C₅ alky, C₁-C₄ alkyl, C₁-C₃ alkyl, C₁-C₂ alkyl or C₁ alkyl, substituted with at least one moiety selected from the group comprising: halogen, hydroxy, and mono- or disubstituted amino.

In preferred embodiments, R¹ a C₁ alkyl substituted with a moiety selected from the group comprising: a fluorine preferably 3 fluorine atoms, hydroxy, and methylamino.

In some preferred embodiments, R¹ is H.

In some preferred embodiments, R¹ is trifluoromethyl.

In some preferred embodiments, R¹ is hydroxymethyl.

In some preferred embodiments, R¹ is methylaminomethyl.

In some embodiments, L is C₁-C₅ alkyl, C₁-C₄ alkyl, C₁-C₃ alkyl C₁-C₂ alkyl or C₁ alkyl.

In some preferred embodiments, L is absent.

In some embodiments, Ar is an optionally substituted 5-membered unsaturated heterocyclic ring selected from the group comprising: a pyrrole, furan, thiophene, imidazole, imidazoline, pyrazole, pyrazoline, oxazole, oxazoline, isoxazole, thiazole, thiazoline, isothiazole, triazole, oxadiazole, thiadiazole, dithiazole, and a tetrazole, or derivatives thereof.

In preferred embodiments, Ar is an optionally substituted 5-membered unsaturated heterocyclic ring selected from the group comprising: a furan, imidazole, and an isoxazole, or derivatives thereof.

In preferred embodiments, Ar comprises a ring selected from the group comprising: a furan of Formula (II); an imidazole of Formula (III); and an isoxazole of Formula (IV); wherein R² is a C₁ alkyl preferably substituted with NH₂ or preferably substituted with a mono- or disubstituted amino preferably comprising methylamino.

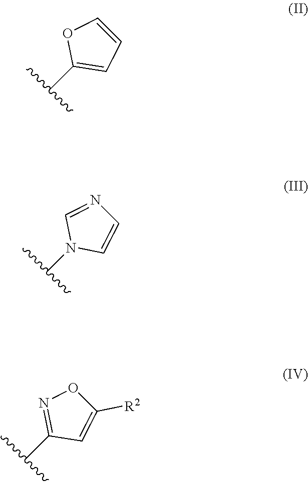

In some preferred embodiments, Ar comprises a furan of Formula (II).

In some preferred embodiments, Ar comprises an imidazole of Formula (III).

In some preferred embodiments, Ar comprises an isoxazole of Formula (IV); wherein R² is a C₁ alkyl preferably substituted with NH₂ or preferably substituted with a mono- or disubstituted amino preferably comprising methylamino. In some embodiments, when X is N, R¹ is a C₁ alkyl substituted with at least one moiety selected from the group comprising: halogen, hydroxy, and mono- or disubstituted amino.

In some embodiments, when Ar is an imidazole or furan, R¹ is a C₁ alkyl substituted with at least one moiety selected from the group comprising: halogen, hydroxy, and mono- or disubstituted amino.

In some embodiments, when Ar is an isoxazole, R¹ is H.

In some embodiments, when Ar is an isoxazole, X is CH.

In some embodiments, when Ar is an isoxazole or furan, L is absent.

In some embodiments, R¹ is H or a straight or branched C₁-C₁₀ alkyl, C₁-C₅ alky, C₁-C₄ alkyl, C₁-C₃ alkyl, C₁-C₂ alkyl or C₁ alkyl, substituted with at least one moiety selected from the group comprising: halogen, hydroxy, and mono- or disubstituted amino; X is CH or N; L is absent or C₁-C₅ alkyl; and Ar is an optionally substituted 5-membered unsaturated heterocyclic ring.

In preferred embodiments, R¹ is H or a C₁ alkyl substituted with a moiety selected from the group comprising: a fluorine preferably 3 fluorine atoms, hydroxy, and methylamino; X is CH or N; L is absent or C₁-C₅ alkyl; and Ar is an optionally substituted 5-membered unsaturated heterocyclic ring.

In some embodiments, R¹ is H or a straight or branched C₁-C₁₀ alkyl, C₁-C₅ alky, C₁-C₄ alkyl, C₁-C₃ alkyl, C₁-C₂ alkyl or C₁ alkyl, substituted with at least one moiety selected from the group comprising: halogen, hydroxy, and mono- or disubstituted amino; X is CH or N; L is absent or C₁-C₅ alkyl; and Ar is an optionally substituted 5-membered unsaturated heterocyclic ring selected from the group comprising: a furan, imidazole, and an isoxazole, or derivatives thereof.

In preferred embodiments, R¹ is H or a C₁ alkyl substituted with a moiety selected from the group comprising: a fluorine preferably 3 fluorine atoms, hydroxy, and methylamino; X is CH or N; L is absent or C₁-C₅ alkyl; and Ar is an optionally substituted 5-membered unsaturated heterocyclic ring selected from the group comprising: a furan, imidazole, and an isoxazole, or derivatives thereof.

In some embodiments, R¹ is H or a straight or branched C₁-C₁₀ alkyl, C₁-C₅ alky, C₁-C₄ alkyl, C₁-C₃ alkyl, C₁-C₂ alkyl or C₁ alkyl, substituted with at least one moiety selected from the group comprising: halogen, hydroxy, and mono- or disubstituted amino; X is CH or N; L is absent or C₁-C₅ alkyl; and Ar comprises a ring selected from the group comprising: a furan of Formula (II); an imidazole of Formula (III); and an isoxazole of Formula (IV); wherein R² is a C₁ alkyl preferably substituted with NH₂ or preferably substituted with a mono- or disubstituted amino preferably comprising methylamino.

In preferred embodiments, R¹ is H or a C₁ alkyl substituted with a moiety selected from the group comprising: a fluorine preferably 3 fluorine atoms, hydroxy, and methylamino; X is CH or N; L is absent or C₁-C₅ alkyl; and Ar comprises a ring selected from the group comprising: a furan of Formula (II); an imidazole of Formula (III); and an isoxazole of Formula (IV); wherein R² is a C₁ alkyl preferably substituted with NH₂ or preferably substituted with a mono- or disubstituted amino preferably comprising methylamino.

In some embodiments, R¹ is H or a straight or branched C₁-C₁₀ alkyl, C₁-C₅ alky, C₁-C₄ alkyl, C₁-C₃ alkyl, C₁-C₂ alkyl or C₁ alkyl, substituted with at least one moiety selected from the group comprising: halogen, hydroxy, and mono- or disubstituted amino; X is CH or N; L is absent or C₁ alkyl; and Ar is an optionally substituted 5-membered unsaturated heterocyclic ring.

In preferred embodiments, R¹ is H or a C₁ alkyl substituted with a moiety selected from the group comprising: a fluorine preferably 3 fluorine atoms, hydroxy, and methylamino; X is CH or N; L is absent or C₁ alkyl; and Ar is an optionally substituted 5-membered unsaturated heterocyclic ring.

In some embodiments, R¹ is H or a straight or branched C₁-C₁₀ alkyl, C₁-C₅ alky, C₁-C₄ alkyl, C₁-C₃ alkyl, C₁-C₂ alkyl or C₁ alkyl, substituted with at least one moiety selected from the group comprising: halogen, hydroxy, and mono- or disubstituted amino; X is CH or N; L is absent or C₁ alkyl; and Ar is an optionally substituted 5-membered unsaturated heterocyclic ring selected from the group comprising: a furan, imidazole, and an isoxazole, or derivatives thereof.

In preferred embodiments, R¹ is H or a C₁ alkyl substituted with a moiety selected from the group comprising: a fluorine preferably 3 fluorine atoms, hydroxy, and methylamino; X is CH or N; L is absent or C₁ alkyl; and Ar is an optionally substituted 5-membered unsaturated heterocyclic ring selected from the group comprising: a furan, imidazole, and an isoxazole, or derivatives thereof.

In some embodiments, R¹ is H or a straight or branched C₁-C₁₀ alkyl, C₁-C₅ alky, C₁-C₄ alkyl, C₁-C₃ alkyl, C₁-C₂ alkyl or C₁ alkyl, substituted with at least one moiety selected from the group comprising: halogen, hydroxy, and mono- or disubstituted amino; X is CH or N; L is absent or C₁ alkyl; and Ar comprises a ring selected from the group comprising: a furan of Formula (II); an imidazole of Formula (III); and an isoxazole of Formula (IV); wherein R² is a C₁ alkyl preferably substituted with NH₂ or preferably substituted with a mono- or disubstituted amino preferably comprising methylamino.

In preferred embodiments, R¹ is H or a C₁ alkyl substituted with a moiety selected from the group comprising: a fluorine preferably 3 fluorine atoms, hydroxy, and methylamino; X is CH or N; L is absent or C₁ alkyl; and Ar comprises a ring selected from the group comprising: a furan of Formula (II); an imidazole of Formula (III); and an isoxazole of Formula (IV); wherein R² is a C₁ alkyl preferably substituted with NH₂ or preferably substituted with a mono- or disubstituted amino preferably comprising methylamino.

In some embodiments, R¹ is H or a straight or branched C₁-C₁₀ alkyl, C₁-C₅ alky, C₁-C₄ alkyl, C₁-C₃ alkyl, C₁-C₂ alkyl or C₁ alkyl, substituted with at least one moiety selected from the group comprising: halogen, hydroxy, and mono- or disubstituted amino; X is CH or N; L is absent; and Ar is an optionally substituted 5-membered unsaturated heterocyclic ring.

In preferred embodiments, R¹ is H or a C₁ alkyl substituted with a moiety selected from the group comprising: a fluorine preferably 3 fluorine atoms, hydroxy, and methylamino; X is CH or N; L is absent; and Ar is an optionally substituted 5-membered unsaturated heterocyclic ring.

In some embodiments, R¹ is H or a straight or branched C₁-C₁₀ alkyl, C₁-C₅ alky, C₁-C₄ alkyl, C₁-C₃ alkyl, C₁-C₂ alkyl or C₁ alkyl, substituted with at least one moiety selected from the group comprising: halogen, hydroxy, and mono- or disubstituted amino; X is CH or N; L is absent; and Ar is an optionally substituted 5-membered unsaturated heterocyclic ring selected from the group comprising: a furan, imidazole, and an isoxazole, or derivatives thereof.

In preferred embodiments, R¹ is H or a C₁ alkyl substituted with a moiety selected from the group comprising: a fluorine preferably 3 fluorine atoms, hydroxy, and methylamino; X is CH or N; L is absent; and Ar is an optionally substituted 5-membered unsaturated heterocyclic ring selected from the group comprising: a furan, imidazole, and an isoxazole, or derivatives thereof.

In some embodiments, R¹ is H or a straight or branched C₁-C₁₀ alkyl, C₁-C₅ alky, C₁-C₄ alkyl, C₁-C₃ alkyl, C₁-C₂ alkyl or C₁ alkyl, substituted with at least one moiety selected from the group comprising: halogen, hydroxy, and mono- or disubstituted amino; X is CH or N; L is absent; and Ar comprises a ring selected from the group comprising: a furan of Formula (II); an imidazole of Formula (III); and an isoxazole of Formula (IV); wherein R² is a C₁ alkyl preferably substituted with NH₂ or preferably substituted with a mono- or disubstituted amino preferably comprising methylamino.

In preferred embodiments, R¹ is H or a C₁ alkyl substituted with a moiety selected from the group comprising: a fluorine preferably 3 fluorine atoms, hydroxy, and methylamino; X is CH or N; L is absent; and Ar comprises a ring selected from the group comprising: a furan of Formula (II); an imidazole of Formula (III); and an isoxazole of Formula (IV); wherein R² is a C₁ alkyl preferably substituted with NH₂ or preferably substituted with a mono- or disubstituted amino preferably comprising methylamino.

In preferred embodiments, the compound is selected from the group comprising: 2-(1H-imidazol-1-yl)-5-(trifluoromethyl)pyridine (M4); 6-(2-furanyl)-N-methyl-3-pyridinemethanamine (M8); 5-[(methylamino)methyl]-3-phenylisoxazole (M14); (3-phenyl-5-isoxazolyl)methanamine (M28); and [4-(1H-imidazol-1-ylmethyl)phenyl]methanol (M38); or a tautomer, isomer, prodrug, metal complex, or pharmaceutically acceptable salt thereof. The structures of these compounds are shown below:

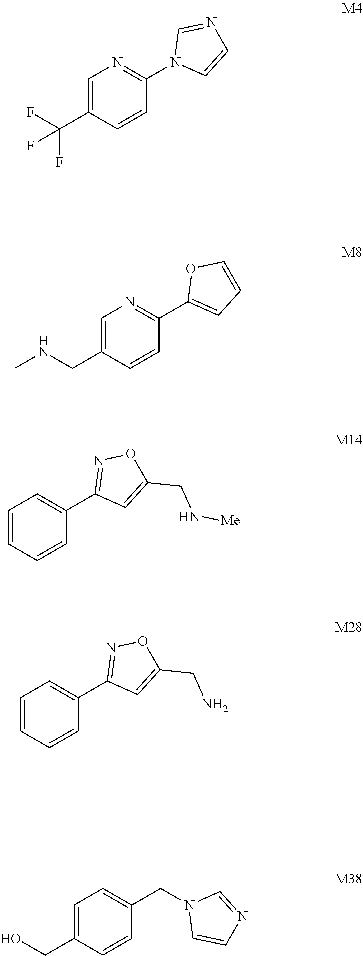

In an especially preferred embodiment, the compound is 6-(2-furanyl)-N-methyl-3-pyridinemethanamine (M8) or a tautomer, isomer, prodrug, metal complex, or pharmaceutically acceptable salt thereof.

The compound may be formulated in a conventional pharmaceutical, cosmetic, or nutritional composition. The composition may be suitable for administration orally, parenterally, topically, or transdermally. The composition may comprise a solid, a capsule, tablet, syrup, injectable solution or suspension, ointment, suppository, controlled-release form, water-soluble granulate. The composition may comprise other active ingredients having complementary or anyway useful activity in addition to the carriers and excipients used in the pharmaceutical technique. The composition may contain cinnamon and/or chromium.

The compound may comprise a dosage of at least 1 mg/kg body weight/day, or of at least 2, 3, 4, 5, 6, 7, 8, 9, 10, 15, 20, 25, 30, 35, or of at least 40 mg/kg body weight/day. The compound may comprise a dosage of no greater than 100 mg/kg body weight/day, or of no greater than 95, 90, 85, 80, 75, 70, 65, 60, 55, or of no greater than 50 mg/kg body weight/day. The compound may preferably comprise a dosage of between 40-50 mg/kg body weight/day.

In some embodiments, the metabolic disorder comprises a weight or dietary-related metabolic disorder. The weight or dietary-related metabolic disorder may be selected from the group comprising: obesity, a glucose-related metabolic disorder, dyslipidaemia, hypertension, and metabolic syndrome.

In preferred embodiments, the metabolic disorder comprises a weight or dietary-related metabolic disorder comprising obesity and/or a glucose-related metabolic disorder.

In some embodiments, the glucose-related metabolic disorder is selected from the group comprising: type-1 diabetes, type-2 diabetes, prediabetes, insulin resistance optionally comprising obesity-induced insulin resistance, impaired glucose tolerance, elevated blood glucose, hyperinsulinemia, and diabetes related diseases.

In preferred embodiments, the glucose-related metabolic disorder comprises insulin resistance and preferably obesity-induced insulin resistance.

In preferred embodiments, the compound may be for use in inducing weight loss in a subject in need of losing weight.

It has been found that 2-(1H-imidazol-1-yl)-5-(trifluoromethyl)pyridine (M4); 6-(2-furanyl)-N-methyl-3-pyridinemethanamine (M8); 5-[(methylamino)methyl]-3-phenylisoxazole (M14); (3-phenyl-5-isoxazolyl)methanamine (M28); and [4-(1H-imidazol-1-ylmethyl)phenyl]methanol (M38); or a tautomer, isomer, prodrug, metal complex, or pharmaceutically acceptable salt thereof are particularly effective against obesity or overweight. These compounds are also effective against glucose-related metabolic disorders comprising insulin resistance and preferably obesity-induced insulin resistance.

According to a second aspect of the invention, there is provided the use of a compound of Formula (I) or a tautomer, isomer, prodrug, metal complex, or pharmaceutically acceptable salt thereof in the manufacture of a medicament for a metabolic disorder or for weight loss induction.

The compound may comprise any compound of the first aspect of the invention.

The metabolic disorder may comprise any metabolic disorder of the first aspect of the invention.

According to a third aspect of the invention, there is provided a method of treating a metabolic disorder in a subject in need of treatment comprising administering to the subject a compound of Formula (I) or a tautomer, isomer, prodrug, metal complex, or pharmaceutically acceptable salt thereof.

The compound may comprise any compound of the first aspect of the invention.

The metabolic disorder may comprise any metabolic disorder of the first aspect of the invention.

The method may comprise administering the compound at a dosage of at least 1 mg/kg body weight/day, or of at least 2, 3, 4, 5, 6, 7, 8, 9, 10, 15, 20, 25, 30, 35, or of at least 40 mg/kg body weight/day. The method may comprise administering the compound at a dosage of no greater than 100 mg/kg body weight/day, or of no greater than 95, 90, 85, 80, 75, 70, 65, 60, 55, or of no greater than 50 mg/kg body weight/day. The method may preferably comprise administering the compound at a dosage of between 40-50 mg/kg body weight/day.

According to a fourth aspect of the invention, there is provided a method of inducing weight loss in a subject in need of losing weight comprising administering to the subject a compound of Formula (I) or a tautomer, isomer, prodrug, metal complex, or pharmaceutically acceptable salt thereof.

The compound may comprise any compound of the first aspect of the invention.

The subject may be overweight or obese.

The subject may be overweight and have a body mass index (BMI) of between 25 and 29.9. The subject may be obese and have a BMI of between 30 and 39.9. The subject may be severely obese and have a BMI of 40 or greater.

The subject may have a waist size of 80 cm or greater, or of 90 cm or greater.

The subject may have a glucose-related metabolic disorder, which may comprise insulin resistance and preferably obesity-induced insulin resistance.

The method may comprise administering the compound at a dosage of at least 1 mg/kg body weight/day, or of at least 2, 3, 4, 5, 6, 7, 8, 9, 10, 15, 20, 25, 30, 35, or of at least 40 mg/kg body weight/day. The method may comprise administering the compound at a dosage of no greater than 100 mg/kg body weight/day, or of no greater than 95, 90, 85, 80, 75, 70, 65, 60, 55, or of no greater than 50 mg/kg body weight/day. The method may preferably comprise administering the compound at a dosage of between 40-50 mg/kg body weight/day.

### DETAILED DESCRIPTION OF THE INVENTION

In order that the invention may be more clearly understood embodiments thereof will now be described, by way of example only, with reference to the accompanying drawings, of which:

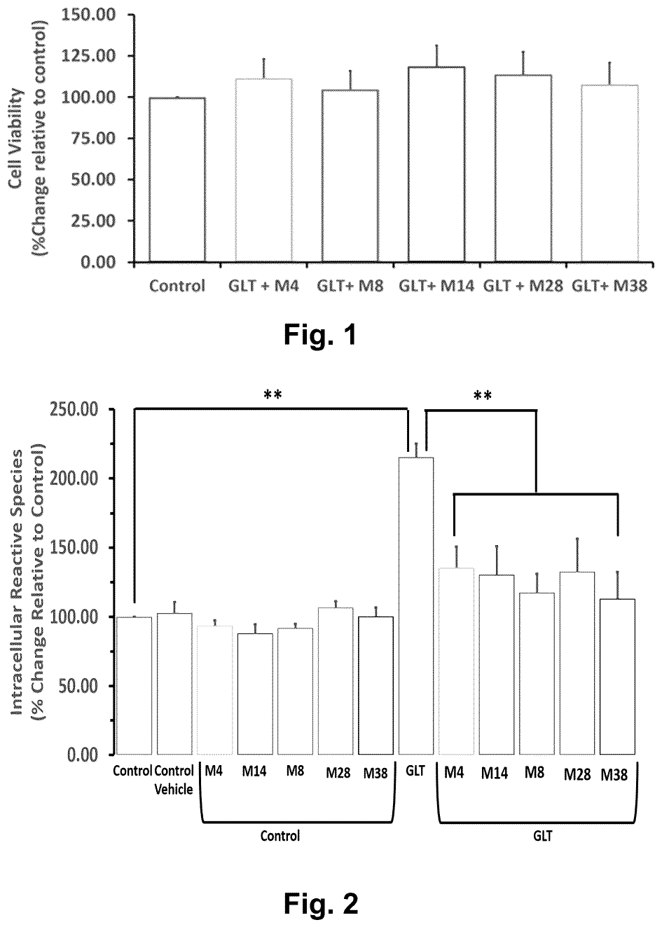

FIG. 1 shows a bar graph displaying cell viability (expressed as a % change relative to control from 3 independent experiments±SEM) of C2C12 skeletal muscle cells following their culture in six different media for 5 days. Bars represent the following media used: Control—a Roswell Park Memorial Institute-1640 (RPMI-1640) control medium; GLT+M4—a glucolipotoxic (GLT) RPMI-1640 medium (28 mM glucose, 200 μM palmitic acid and 200 μM oleic acid) with 100 μM of added 2-(1H-imidazol-1-yl)-5-(trifluoromethyl)pyridine (M4); GLT+M8—GLT RPMI-1640 medium with 100 UM of added 6-(2-furanyl)-N-methyl-3-pyridinemethanamine (M8); GLT+M14—GLT RPMI-1640 medium with 100 μM of added 5-[(methylamino)methyl]-3-phenylisoxazole (M14); GLT+M28—GLT RPMI-1640 medium with 100 μM of added (3-phenyl-5-isoxazolyl)methanamine (M28); and GLT+M38—GLT RPMI-1640 medium with 100 μM of added [4-(1H-imidazol-1-ylmethyl)phenyl]methanol (M38). The graph shows that none of the tested compounds (M4, M8, M14, M28, and M38) reduce C2C12 skeletal muscle cell viability.

FIG. 2 shows a bar graph displaying reactive species scavenging abilities of five compounds (2-(1H-imidazol-1-yl)-5-(trifluoromethyl)pyridine (M4); 5-[(methylamino)methyl]-3-phenylisoxazole (M14); 6-(2-furanyl)-N-methyl-3-pyridinemethanamine (M8); (3-phenyl-5-isoxazolyl)methanamine (M28); and [4-(1H-imidazol-1-ylmethyl)phenyl]methanol (M38)) that were independently used to treat for 1 h periods C2C12 skeletal muscle cells that had been cultured in control RPMI-1640 or GLT RPMI-1640 media for 5 days. The graph shows intracellular reactive species (expressed as a % change relative to control from 4 independent experiments±SEM; ** represents a change with p<0.005). Bars represent from left to right: Control—cells cultured in control medium with no additional compounds added; Control Vehicle—cells cultured in a 1% ethanol solution; Control+inventive compound—cells cultured in control medium and treated with one of the five compounds; GLT—cells cultured in GLT medium with no additional compounds added; and GLT+inventive compound—cells cultured in GLT medium and treated with one of the five compounds. The graphs show that exposure of cells to GLT media results in significantly enhanced presence of reactive species within the cells. Independent incubation in the presence of the compounds reduces GLT-associated reactive species levels and in some cases close to control values.

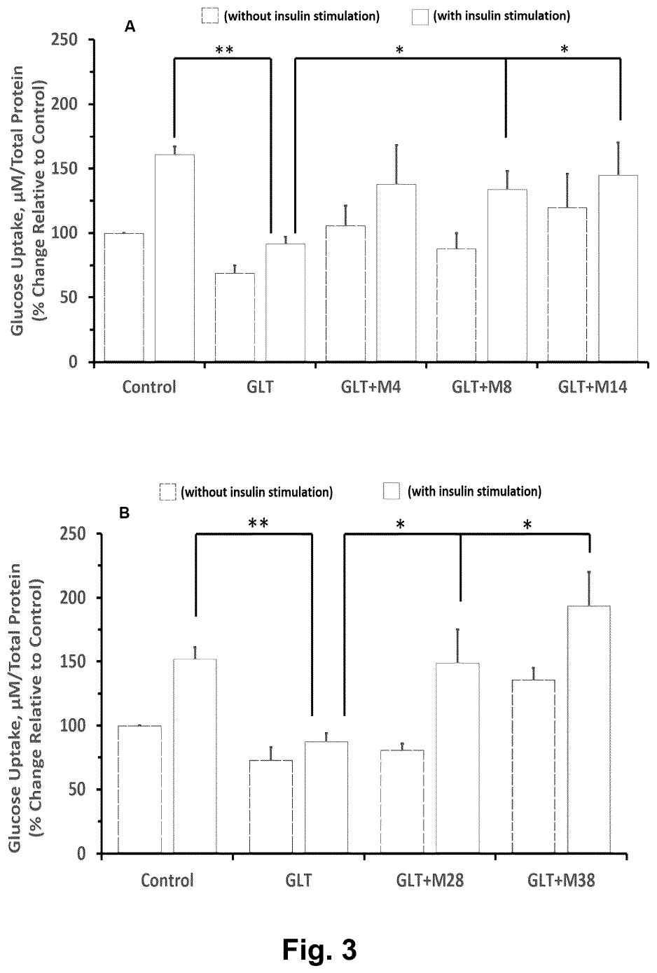

FIG. 3 shows bar graphs A and B displaying glucose uptake in C2C12 skeletal muscle cells treated with selected compounds. The graphs show glucose uptake (μM) per total cellular protein of C2C12 skeletal muscle cells that were cultured for 5 days in Dulbecco's Minimal Eagle's Medium (DMEM) media or in DMEM GLT media. Data is shown for stimulated cells that were treated for 1 h with 100 nM of insulin as well as for unstimulated cells. Bars represent the following media used: Control—a control DMEM medium; GLT-GLT DMEM medium; GLT+M4—GLT DMEM medium supplemented with 100 μM of (2-(1H-imidazol-1-yl)-5-(trifluoromethyl)pyridine (M4); GLT+M8—GLT DMEM medium supplemented with 100 μM of 6-(2-furanyl)-N-methyl-3-pyridinemethanamine (M8); GLT+M14—GLT DMEM medium supplemented with 100 μM of 5-[(methylamino)methyl]-3-phenylisoxazole (M14); GLT+M28—GLT DMEM medium supplemented with 100 μM of (3-phenyl-5-isoxazolyl)methanamine (M28); and GLT+M38—GLT DMEM medium supplemented with 100 μM of [4-(1H-imidazol-1-ylmethyl)phenyl]methanol (M38). Data are expressed as mean±SEM from 3 or more independent experiments. ** represents a change with p<0.005 and * represents a change with p<0.05. The graph demonstrates the ability of the compounds to reverse glucolipotoxic inhibition of insulin-stimulated glucose uptake, returning values close to, or even above, control values.

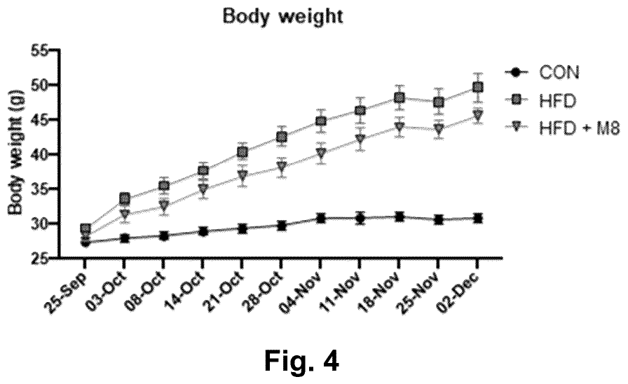

FIG. 4 shows a graph of body weight (g) versus time of obese high fat-fed mice. The graph contains three curves: a Control curve wherein the mice were kept on a normal diet and were not high fat-fed; an HFD curve wherein the mice were kept on a high fat diet without administration of any other compound; and an HFD+M8 curve wherein the mice were kept on a high fat diet and were also administered 6-(2-furanyl)-N-methyl-3-pyridinemethanamine (M8). The graph shows that administration of M8 allows for a significant reduction of body weight in obese high fat-fed mice.

### EXAMPLES

All experiments were approved by a local ethical review committee and carried out under UK Home Office approval and according to the Animals Scientific Procedures Act (1986).

### Toxicity Testing of Compounds of the Invention

C2C12 muscle cell myotubes were cultured for 5 days in the following four culture media: a control RPMI-1640 medium; glucolipotoxic (GLT) RPMI-1640 medium (28 mM glucose, 200 μM palmitic acid and 200 μM oleic acid) supplemented with 100 μM of M4; GLT RPMI-1640 medium supplemented with 100 M of M8; GLT RPMI-1640 medium supplemented with 100 μM of M14; GLT RPMI-1640 medium supplemented with 100 μM of M28; and GLT RPMI-1640 medium supplemented with 100 μM of M38. Following culture, media were aspirated and cells washed 3 times in Krebs-Ringer buffer (KRB). A final concentration of 5 M Calcein AM Cell Viability Dye (ThermoFischer) in KRB was loaded for 1 h before washing again with KRB. Cell viability was measured via fluorescence, with excitation and emission at 490 nm and 520 nm, respectively.

### Results of Toxicity Testing of Compounds of the Invention

The results of the compound toxicity testing, as displayed in FIG. 1, show that none of the five compounds of the invention (M4, M8, M14, M28, and M38) reduce C2C12 skeletal muscle cell viability and are thus non-toxic to the cells.

### Reactive Species Scavenging Ability of Compounds of the Invention

C2C12 muscle cell myotubes were cultured for 5 days in standard RPMI-1640 tissue culture media or GLT RPMI-1640 media. Corresponding media were independently supplemented with 100 μM of a compound (M4, M14, M8, M28, and M38 investigated). Non-supplemented standard and GLT RPMI-1640 media were also retained as controls. Cells were thereafter washed 3 times in KRB and 20 μM 2′,7′-dichlorofluorescein diacetate (DCFDA), a cell permeant fluorogenic dye that measures hydroxyl, peroxyl and other ROS, was loaded for 1 h. Reactive species detection was measured via fluorescence, with excitation at 495 nm and emission at 530 nm.

### Results of Reactive Species Scavenging Ability of Compounds of the Invention

The results of the compounds' reactive species scavenging testing, as displayed in FIG. 2, show that exposure to glucolipotoxic (GLT) media results in significantly enhanced presence of damaging reactive species within cells.

Incubation in the presence of the indicated compound of the invention reduced GLT-associated reactive species levels by ˜75% and in some cases back down to non-GLT control levels.

The reactive species scavenging ability of the compounds of the invention potentially confers significant clinical benefit to use of the compounds as therapeutics to treat diseases associated with metabolic stress. For instance, the ability to scavenge glycation and lipidation end-products allows for restoration of normal cellular function in key tissues associated with insulin secretion and insulin resistance.

Glucose Uptake in C2C12 Skeletal Muscle Cells Treated with Compounds of the Invention

C2C12 skeletal muscle cell myotubes were cultured for 5 days in standard DMEM tissue culture media or GLT DMEM media. The media were either used without further supplementation (used as controls) or were independently supplemented with 100 μM of a compound (M4, M8, M14, M28, and M38 investigated). Myotubes were then serum-starved overnight in DMEM supplemented with 5 mM glucose, and thereafter incubated for 1 h in glucose-free DMEM or in glucose-free DMEM supplemented with 100 nM insulin stimulant. The media were thereafter replaced with phosphate buffered saline (PBS) containing 0.125 mM 2-deoxy glucose (2-DG). Glucose uptake reactions were conducted for 30 min, and then terminated by addition of stop buffer (0.4 M HCl+2% dodecyl trimethyl ammonium bromide). 2-deoxyglucose-6-phosphate (2DG6P) detection reagent was applied, and data were acquired using a CLARIOStar luminometer (BMG Labtech, Ortenberg, Germany).

Results of Glucose Uptake in C2C12 Skeletal Muscle Cells Treated with Compounds of the Invention

The results of glucose uptake in C2C12 skeletal muscle cells treated with compounds, as displayed in FIG. 3, show that 5-day exposure of the cells to GLT media results in significant reduction of insulin-stimulated glucose uptake.

Incubation of the cells in the presence of the indicated compound significantly increases insulin sensitivity in cells exposed to glucolipotoxicity (GLT), returning insulin-stimulated glucose uptake close to, or above, control values.

The 5-day exposure of C2C12 skeletal muscle cells to GLT media provides for a cellular model of obesity and diabetes. Obesity is associated with the development of insulin resistance, which results in skeletal muscle cells failing to respond to extracellular insulin and taking insufficient glucose into cells for use as an energy source. Obesity-induced insulin resistance increases demand on the pancreas to release more insulin. This can lead to type-2 diabetes, which is characterised by a significant reduction in insulin secretion caused by pancreatic dysfunction, which arises as the pancreas struggles to address a sustained demand for increased insulin secretion placed upon it. The ability of the compounds to reverse GLT inhibition of insulin stimulated glucose uptake is beneficial to the control of glucose homeostasis and the control of blood sugar levels which could result in a reduced risk of developing diseases such as type-2 diabetes, obesity, Metabolic Syndrome, and other associated diseases where cells and tissues are under sustained metabolic stress.

### Effect of M8 on Body Weight in an In Vivo Model of Obesity

High fat-fed mice were used as an animal model of obesity. The body weights of the mice were monitored over a period of 10 weeks. The following three sets of mice were used in the study:
Non-high fat-fed mice that were administered a normal diet.
High fat-fed mice that were administered a high fat diet.
High fat-fed mice that were administered a high fat diet alongside 45 mg/kg body weight/day of compound M8, which was administered via drinking water.

High fat-fed mice were fed the following high fat diet from Research Diets: (60% fat; D12492). Non-high fat-fed mice were used as a control and were fed the following low fat diet from Research Diets: (10% fat, D12450B). The body weights of the mice were measured throughout.

### Results of the Effect of M8 on Body Weight in an In Vivo Model of Obesity

The results of the in vivo study, as displayed in FIG. 4, demonstrate that M8 significantly reduces the body weight of the mice. The body weights at the end of the study of high fat-fed mice that were administered M8 are significantly lower than the corresponding high fat-fed mice that were not administered M8.

Furthermore, M8 displayed no toxic effects on the animals.

The results demonstrate the physiological benefit and efficacy of the hydrolytically stable compounds in reducing body weight in living animals. The compounds are therefore useful in the treatment of weight or dietary-related metabolic disorders, such as obesity. This could also potentially help reduce risk of developing diseases such as type-2 diabetes, Metabolic Syndrome, cancer, and other diseases where obesity has been linked to the development of disease pathophysiology.

The above embodiments are described by way of example only. Many variations are possible without departing from the scope of the invention as defined in the appended claims.

1. A compound of Formula (I) or a tautomer, isomer, prodrug, metal complex, or pharmaceutically acceptable salt thereof for use in the treatment of a metabolic disorder or for inducing weight loss: 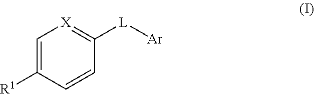
wherein R¹ is:
H; a straight or branched alkyl; a straight or branched alkyl substituted with at least one moiety selected from the group comprising: halogen, hydroxy, alkoxy, NH₂, mono- or disubstituted amino, thiol, and phosphine; aryl; aralkyl; hydroxy; alkoxy; aryloxy; arylalkoxy; NH₂; mono- or disubstituted amino; or halogen;
X is:
CH or N;
L is absent or present, and when present is:
an optionally substituted straight or branched C₁-C₁₀ alkyl, optionally containing one or more rings, and/or optionally containing one or more double bonds; and
Ar is:
an optionally substituted 5-membered unsaturated heterocyclic ring.

2. A compound for use as claimed in claim 1, wherein R¹ is a straight or branched C₁-C₁₀ alkyl, preferably substituted with at least one moiety selected from the group comprising: halogen, hydroxy, and mono- or disubstituted amino.

3. A compound for use as claimed in claim 2, wherein R¹ is a C₁ alkyl, preferably substituted with at least one moiety selected from the group comprising: halogen, hydroxy, and mono- or disubstituted amino.

4. A compound for use as claimed in claim 3, wherein R¹ is a C₁ alkyl, preferably substituted with a moiety selected from the group comprising: a fluorine preferably 3 fluorine atoms, hydroxy, and methylamino.

5. A compound for use as claimed in claim 1, wherein L is C₁-C₅ alkyl.

6. A compound for use as claimed in claim 5, wherein L is C₁ alkyl.

7. A compound for use as claimed in claim 1, wherein Ar is an optionally substituted 5-membered unsaturated heterocyclic ring selected from the group comprising: a pyrrole, furan, thiophene, imidazole, imidazoline, pyrazole, pyrazoline, oxazole, oxazoline, isoxazole, thiazole, thiazoline, isothiazole, triazole, oxadiazole, thiadiazole, dithiazole, and a tetrazole, or derivatives thereof.

8. A compound for use as claimed in claim 7, wherein Ar is an optionally substituted 5-membered unsaturated heterocyclic ring selected from the group comprising: a furan, imidazole, and an isoxazole, or derivatives thereof.

9. A compound for use as claimed in claim 8, wherein Ar comprises a ring selected from the group comprising: a furan of Formula (II); an imidazole of Formula (III); and an isoxazole of Formula (IV); wherein R² is a C₁ alkyl preferably substituted with NH₂ or preferably substituted with a mono- or disubstituted amino preferably comprising methylamino.

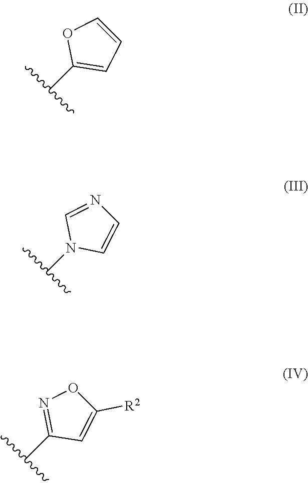

10. A compound for use as claimed in claim 1, wherein the compound is selected from the group comprising: 2-(1H-imidazol-1-yl)-5-(trifluoromethyl)pyridine; 6-(2-furanyl)-N-methyl-3-pyridinemethanamine; 5-[(methylamino)methyl]-3-phenylisoxazole; (3-phenyl-5-isoxazolyl)methanamine; and [4-(1H-imidazol-1-ylmethyl)phenyl]methanol; or a tautomer, isomer, prodrug, metal complex, or pharmaceutically acceptable salt thereof.

11. A compound for use as claimed in claim 10, wherein the compound is 6-(2-furanyl)-N-methyl-3-pyridinemethanamine or a tautomer, isomer, prodrug, metal complex, or pharmaceutically acceptable salt thereof.

12. A compound for use in the treatment of a metabolic disorder as claimed in claim 1, wherein the metabolic disorder comprises a weight or dietary-related metabolic disorder.

13. A compound for use in the treatment of a metabolic disorder as claimed in claim 12, wherein the weight or dietary-related metabolic disorder is selected from the group comprising: obesity, a glucose-related metabolic disorder, dyslipidaemia, hypertension, and metabolic syndrome.

14. A compound for use in the treatment of a metabolic disorder as claimed in claim 13, wherein the weight or dietary-related metabolic disorder comprises obesity and/or a glucose-related metabolic disorder.

15. A compound for use in the treatment of a metabolic disorder as claimed in claim 14, wherein the glucose-related metabolic disorder comprises insulin resistance and preferably obesity-induced insulin resistance.

16. A compound for use as claimed in claim 1, wherein the use comprises inducing weight loss in a subject in need of losing weight.

17. Use of a compound of Formula (I) as defined in claim 1 or a tautomer, isomer, prodrug, metal complex, or pharmaceutically acceptable salt thereof in the manufacture of a medicament for a metabolic disorder or for weight loss induction.

18. A method of treating a metabolic disorder in a subject in need of treatment comprising administering to the subject a compound of Formula (I) as defined in claim 1 or a tautomer, isomer, prodrug, metal complex, or pharmaceutically acceptable salt thereof.

19. A method of inducing weight loss in a subject in need of losing weight comprising administering to the subject a compound of Formula (I) as defined in claim 1 or a tautomer, isomer, prodrug, metal complex, or pharmaceutically acceptable salt thereof.
